(19) United States
(12) Patent Application Publication
(10) Pub. No.: US 2005/0230638 A1
(43) Pub. Date: Oct. 20, 2005

(54) Sterilizer for small items used by babies and children

(75) Inventors: Bruce Ancona, New York, NY (US); Louis Henry, Scarsdale, NY (US)

Correspondence Address: NORMAN H. ZIVIN;Cooper & Dunham LLP, 1185 Avenue of the Americas, New York, NY, 10036, US

(73) Assignee: Kash 'N Gold, Ltd.

(21) Appl. No.: 10/824,143

(22) Filed: Apr. 14, 2004

(51) Int. Cl. A61L 2/10

(52) U.S. Cl. 250/455.11; 422/24

(57) ABSTRACT

A sterilizer for small items which sterilizes items using ultraviolet light in the UV-C range. The sterilizer features a housing which encloses a removable basket or other receptacle made of a material or structure which is ‘transparent’ to UV-C radiation for enclosing items to be sterilized, a motor or other means for turning the receptacle inside the housing, a source of UV light, a door to seal the housing and prevent the leakage of UV light and means for moving the receptacle inside the housing and to activate the UV source. The UV light is directed through the basket and onto the items to be sterilized. As the basket is moved along a rotational axis, the items to be cleaned are continuously moved, insuring that UV light hits all surfaces of each item to be sterilized. The UV light destroys microorganisms on the surfaces of the items to be sterilized. The items are removed from the device in a sterilized state.

### FIELD OF THE INVENTION

The present invention is generally directed to a novel device for sterilizing toys, pacifiers, bottles and other small items that typically come into contact with babies and children. The invention provides a safe, fast and convenient way to clean such items, including those which are not suitable for cleaning or sterilizing by traditional methods, such as hand or machine washing. The device permits exposure of items to ultraviolet light, and provides a means for moving the items to insure that all surfaces of the items are exposed to UW light.

### BACKGROUND OF THE INVENTION

Babies and children often place various items, including toys and game pieces, in their mouths. They also tend to place their own hands or fingers in their mouths, and then use those hands to touch other items. Babies and children also often place the toys, pacifiers, bottles and similar items of other babies and children in their own mouths. When babies and children are sick, they can sneeze or cough onto their toys, game pieces, bottles, and pacifiers, or wipe their noses on their hands, then touch such items. In groups of two or more babies or children, such tendencies can lead to the transfer of harmful bacteria, viruses and other microorganisms from one baby or child to another. Thus, it is important to sterilize all such items before access by another child, particularly in day care centers, doctors' offices, play areas, schools and other places where groups of children are given access to the same toys, games and other items. However, it often is impractical or impossible to thoroughly sterilize all such items through hand or machine washing or other methods that require immersion in water, due to the size, material and/or shapes of such items. Washing such items with a liquid also is not desirable as soaps, chemicals and other agents necessary to disinfect such items, can remain on the surface of the item and be ingested by a baby or child who places the item in his or her mouth after cleaning. Further, some items that are washable in water require significant drying time. Moreover, it is important to insure thorough and complete sterilization of such items. Heat, as from steam or microwaving, is also undesirable as it may damage softer plastics, require cool-down time before re-use, or burn the user. Metal parts found commonly in toy assemblies, such as screws and springs, make microwave activated sterilization impossible. Further, hollows and crevices in toys can retain water when washed in dishwashers, by hand washing or using steam.

The present invention addresses the need for a means of sterilizing toys and other items children contact; a) without damaging the item; b) which can thoroughly and completely sterilize the item; and c) which can sterilize the items without leaving behind chemicals or other compounds which create a hazard for babies and children who will use the item after it is sterilized.

The present invention attains each of these goals through the use, of a compact and convenient sterilizer which uses ultraviolet, or. “UV” light. UV light is invisible radiation having a wavelength of between 100 to 320 nm. The most effective range for sterilization is within 200 nm to 290 nm, designated the UV-C band, with a bandwidth between 250 nm and 260 nm being optimal. At this wavelength, such light is capable of inactivating and destroying a variety of bacteria, viruses, mold and other microorganisms. When UV-C light hits a microorganism, the light is absorbed by the microorganism, which is destroyed. Because the items are sterilized by light, they are not wet by, or submerged in, water, soap or chemicals. Thus, no drying is required, the items are not exposed to extreme heat and no potentially harmful chemicals or soaps are left behind on the item. Even items that are not suitable to be washed in water, such as wood, can be sterilized with UV light.

### SUMMARY OF THE INVENTION

The present invention is directed to a novel device which comprises a housing, made of material suitable to contain UV radiation, which housing encloses a removable basket or other receptacle made of a material or structure which is ‘transparent’ to UV-C radiation, through which UV light may be transmitted, for enclosing items to be sterilized, a motor or other means for turning the receptacle inside the housing, a source of UV light, a door to seal the housing and prevent the leakage of UV light and means for moving the receptacle inside the housing and to activate the UV source. When the system is activated, the motor turns the basket containing items to be sterilized. The UV light is directed through the basket and onto the items to be sterilized. As the basket is moved along a rotational axis, the items to be sterilized are continuously moved, insuring that UV light hits all surfaces of each item to be sterilized. The UV light destroys microorganisms on the surfaces of the items to be sterilized, but does not remain in or on the items. The items are removed from the device in a sterilized state.

### BRIEF DESCRIPTION OF THE DRAWINGS

FIG. 1 shows a front view of an embodiment of the invention.

FIG. 2 shows an exploded view of an embodiment of the invention.

FIG. 3 shows a side view of an embodiment of the invention.

FIG. 4 shows a top view of an embodiment of the invention.

FIG. 5 shows a longitudinal cross-sectional view of the embodiment of FIG. 1 taken approximately along the line A-A of FIG. 1.

FIG. 6 shows a longitudinal cross-sectional view of the embodiment of FIG. 3 taken approximately along the line B-B of FIG. 3.

FIG. 7 shows an embodiment of the device with the basket visible therein.

### DETAILED DESCRIPTION OF THE PREFERRED EMBODIMENTS

Reference will now be made in detail to the preferred embodiments of the invention, examples of which are also provided in the following description. Exemplary embodiments of this invention are described in some detail, although it will be apparent to those skilled in the relevant art that some features that are not particularly important to an understanding of the invention may not be shown for the sake of clarity.

The present invention is directed to a sterilizer for small items used by babies and children. The housing of the sterilizer is constructed of material that is impervious to UV-C radiation, such as plastic. The housing is sealed by a door, which provides access inside the housing. The housing comprises a basket for placing articles to be cleaned, a UV light source, a power source, a motor for rotating such basket and a power switch. The interior of the housing is coated with UV-C material by vacuum coating or electro-coating or is made of UV-C reflective material such as polished aluminum or stainless steel, to reflect UV-C light within the unit and increase exposure of the items therein to UV-C light.

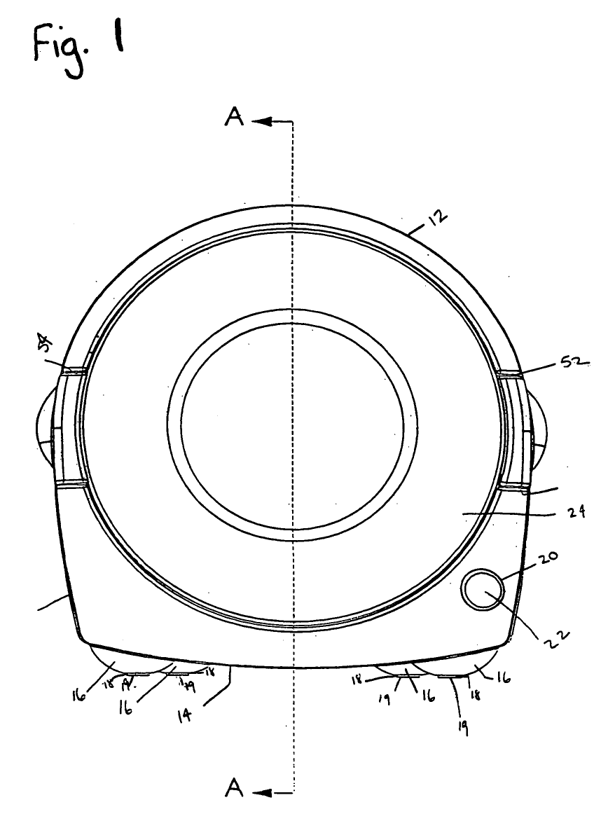

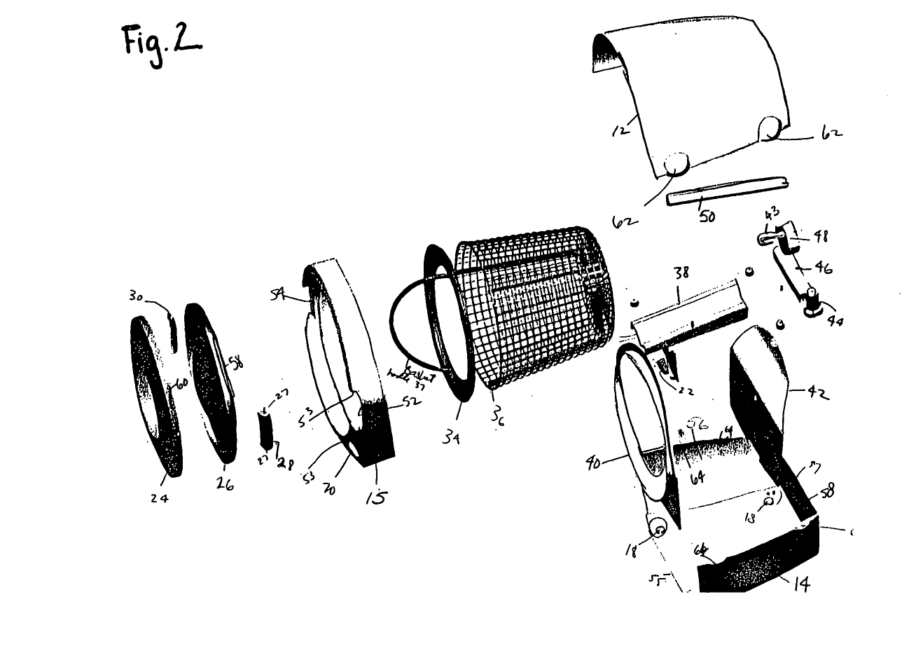

FIG. 1 is a front view of a preferred embodiment of sterilizer 10 comparing a semi-circular shaped cover or dome 12, which accommodates a circular internal basket, and a stable base 14. Four feet 16 are located on each corner of base 14 to provide additional stability, as well as ventilation under the sterilizer 10 and to prevent scratches or markings to surfaces on which the sterilizer 10 is placed. Feet 16 are preferably made of rubber or relatively smooth plastic. Feet 16 have threaded holes extending longitudinally through their centers and are secured to the base 14 by fasteners 18, which preferably are plastic screws, such that the head of each screw is flush with the bottom of foot 16 when fully screwed into foot 16. Felt disks 19 are attached, preferably by glue or other suitable adhesive, to plastic screws 18 to provide additional stability and to provide additional protection to surfaces on which sterilizer 10 are placed. In FIGS. 1 and 2, feet 16 are shown as semi-spherical. However, any shape having flat top and bottom surfaces is suitable. Base 14 accommodates faceplate 15 which is partially semi-circular and has two notches 52 and 54. Notch 52 receives hinge 28 which movably attaches door 24 to faceplate 15. Handle 30 is attached to faceplate 15 at notch 54 and provides means for opening and closing door 24. Faceplate 15 contains hole 20 through which power button 22 can be accessed. Base 14 is shaped to accommodate faceplate 15. Faceplate 15 is shaped to accommodate door 24. In a preferred embodiment door 24 is round. Also in a preferred embodiment, door 24 contains window 26 which is covered with transparent plastic, which filters UV-C rays. UV-C rays can be harmful to eyes and skin, but are blocked by most materials including clear plastics. Thus, window 26 prevents harmful UV-C rays from escaping sterilizer 10 while enabling a user to watch the sterilization process. In a preferred embodiment the transparency of window 26 creates a “violet light” effect when UV-C light source 50 is activated.

FIG. 2 is an exploded view of the sterilizer showing the main components: door 24, window 26, hinge 28, handle 30, front plate 15 having hole 20 preferably located in a lower corner, basket collar 34, basket 36, base 14, track 38, rim 40, back support 42, starter 44, ballast 46, motor 48,; UV-C light source 50 and dome 12. As seen in FIG. 2, base 14 comprises bottom 55, side walls 56 and back panel 57 that further comprises lips 58. Base 14 further comprises flanges 64.

In assembly, window 26 is fitted into door 24 and secured by lips 58, located on both sides of window 26. Lips 58 are pushed under grooves 60 extending along the rear edges of door 24. Window 26 also can be secured to door 24 using readily available adhesives. Door 24 having, in one embodiment, window 26 attached therein is movably attached at one side to faceplate 15 via hinge 28. Hinge 28 is attached at one side to door 24 using standard screws or other fasteners. Hinge 28, attached to door 24, and then is seated into notch 52. Spring-loaded protrusions 27 in hinge 28 fit into holes 53 drilled into the top and bottom of notch 52, enabling hinge 28 and door 24 to move in an arc of about 180 degrees. While the arc may be decreased, an arc of about 180 degrees allows for ease of filling, emptying, removal and replacement of basket 36 when the sterilizer 10 or basket 36 is being cleaned, serviced, filled or emptied. Handle 30 is attached to door 24 at a point on the rim of door 24 that is across from the point at which hinge 27 is attached to door 24. Handle 30 is attached to door 24 using standard fasteners such as screws. Handle 30 comprises a spring mechanism or other locking mechanism that holds door 24 securely to faceplate 15 when the sterilizer 10 is in a closed position. Rim 40, which preferably is made of rubber, is seated into the rear of faceplate 15 such that a seal is formed around door 24 when the sterilizer 10 is in a closed position, preventing leakage of UV-C radiation when the unit is operating. Basket collar 34 is rigidly attached to basket 36. Basket handle 37 is movably attached to basket collar 34 such that it pivots to lie along basket collar 34 when not in use. Track 38 is fastened to the inside surface of base 14 using standard screws such that a space of about 1 inch is created between base 20 and the roof of track 38. Power button 22 is placed through hole 20 from the internal face of faceplate 15, such that power button 20 protrudes from the front of faceplate 15.

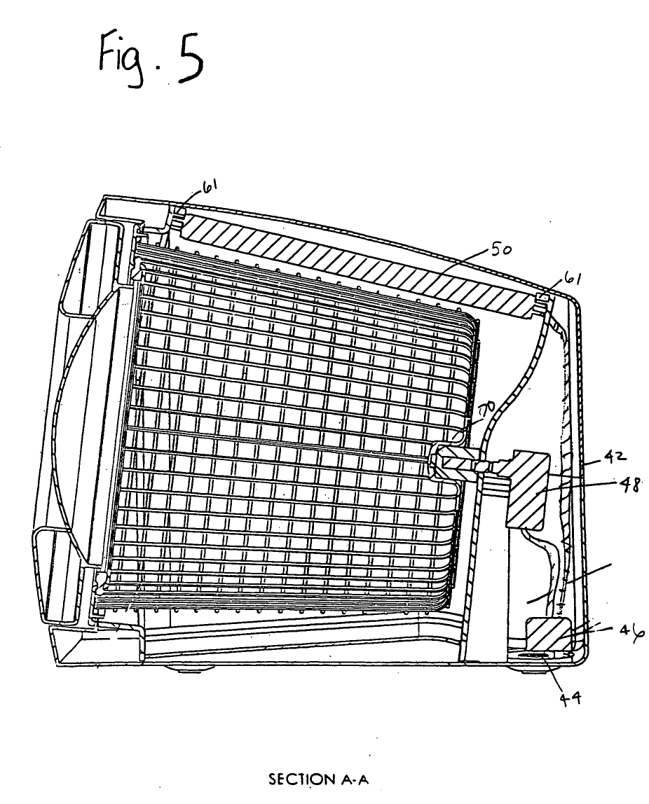

As can more easily be seen in FIG. 5, which is a longitudinal sectional view of the embodiment of FIG. 1 taken approximately along the line A-A of FIG. 1, when assembled, motor 48 is attached by standard fasteners such as screws to the center of back support 42. Sprocket 43 on motor 42 engages the ribs of well 70, formed in the bottom of basket 36. As also seen in FIG. 5, UV-C light source 50 is placed into sockets 61 fastened inside dome 12.

In a preferred embodiment, UV-C light source 50 is cylindrical, type G6T5, has wattage of 6, a base diameter of 15 mm, lamp diameter of 15 mm, is 9 inches in length, has 2 2-pin aluminum bases and is constructed of hard glass quartz. Effective sources of UV-C light are low pressure mercury discharge lamps. A preferred lamp is manufactured by Royal Philips Electronics. Another preferred lamp is manufactured by Osram Sylvania, Inc. Intensity at 1 meter using a type G6T5 bulb preferably is 16.7 uW/cm². In a preferred embodiment, starting voltage of UV-C source 50 is 120 VAC @ 60 HZ, operational voltage is 120VAC @ 60 HZ, UV output is 253.7 nm @ 100 hrs. The average lamp life of a G6T5-type is 6,000 hours.

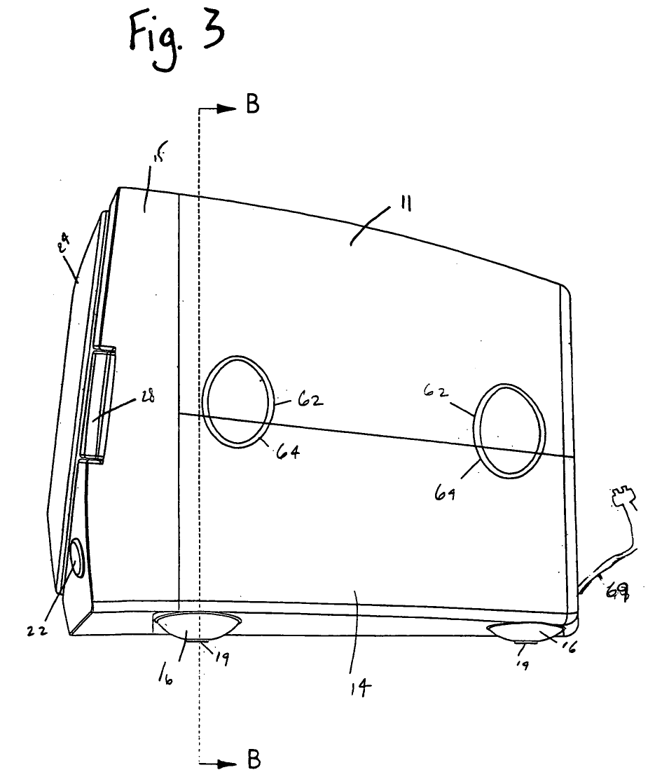

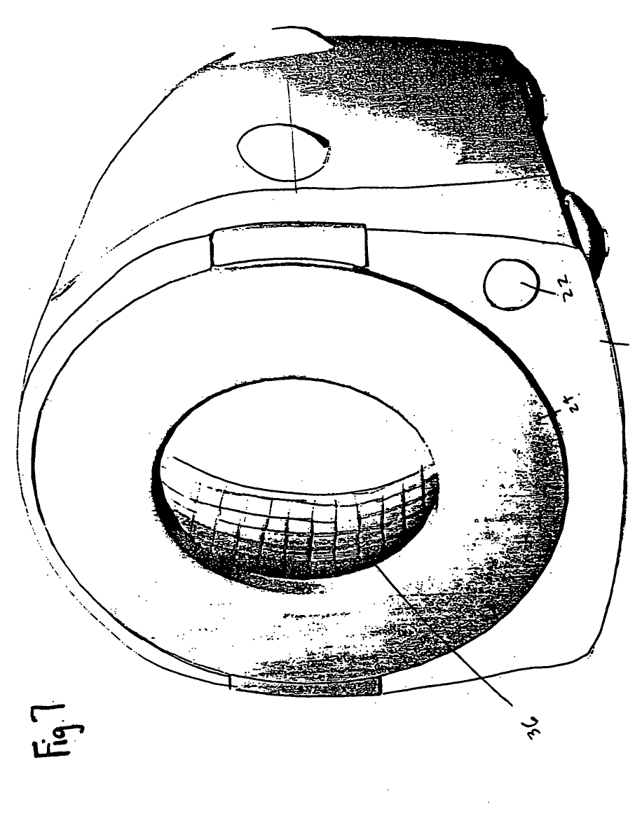

As best seen in FIG. 7, faceplate 15, which is attached to door 24, window 26, handle 30 and hinge 28 as described above, is attached by standard means such as screw means 62 to dome 12. As seen in FIG. 3, in a preferred embodiment, faceplate 15 is angled toward the user, enabling the sterilizer to be accessed easily from a shelf or the floor. Housing 15 is attached to base 14 such that flanges 62 on dome 12 contact flanges 64 on base 14. Flanges 62 and 64 receive screws to facilitate fastening of housing 15 to dome 12.

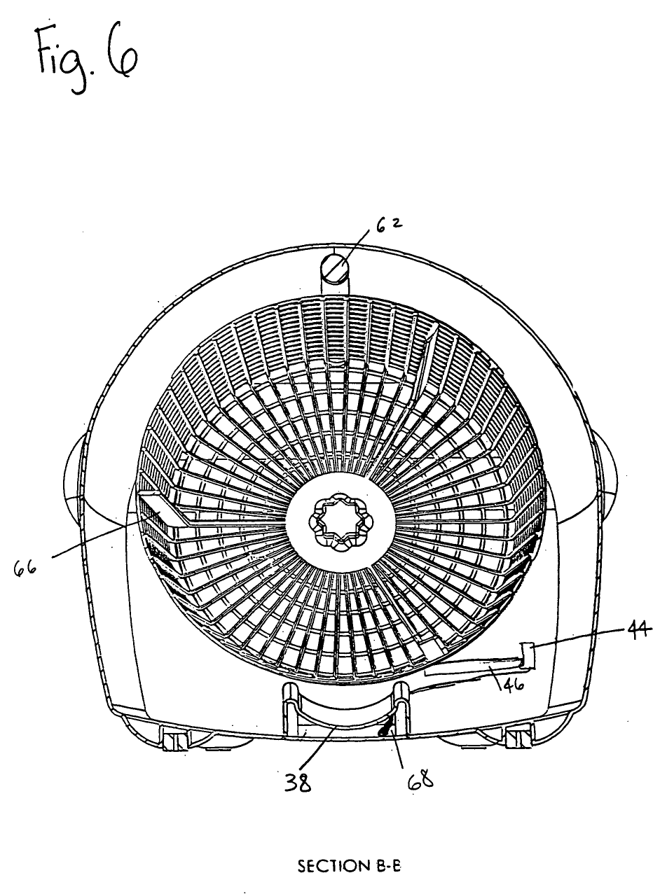

As seen in FIG. 6, basket 36 is slid into sterilizer 10 along track 38. Basket 36 has paddles 66 protruding from several points of its internal wall to encourage movement of items in basket 36 when the sterilizer 10 is in use. Basket 36 has a proliferation of openings throughout to facilitate exposure of items therein to UV-C light so that sterilization takes place in an optimum amount of time. Alternatively, basket 36 is made of a transparent plastic mesh.

As best seen in FIG. 6, wire 68 is attached to starter 44 and runs under track 38. Wire 68 is attached at the opposite end to power button. 22. As best seen in FIG. 5, starter 44 is electrically attached to ballast 46 which provides resistance to stabilize current in the circuit created when sterilizer 10 is attached to a power source via power cord 69. In a preferred embodiment, ballast 46 is operational with 100/200VAC at 50/60 HZ.

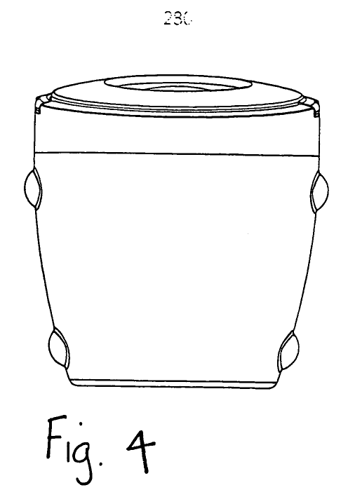

As can be seen in FIG. 4, in one embodiment the overall design of sterilizer 10 is compact and slightly narrower at the rear, enabling it to be placed on a shelf or counter.

In operation, door 24 is opened using handle 30, and items to be sterilized are placed into basket 36. Door 24 is pivoted on hinge 28 into a closed position. Power button 22 is pressed, closing the electrical circuit formed by starter 44, motor 48 and UV-C light source 50. Motor 48 is activated and its shaft turns in a circular path, causing sprocket 43 to engage well 60, thereby causing basket 36 to turn in a circular path. Items in basket 36 are randomly moved inside basket 36. The items in basket 36 randomly strike paddles 66 and are further moved randomly inside basket 36. Activation of the electrical circuit also causes UV-C light source 50 to be activated and to emit ultraviolet light in a direction toward basket 36. As basket 36 spins, the items inside it are exposed to the UV-C light emitted from light source 50. The random movement of the items inside basket 36 increases the likelihood that all surfaces of the items inside basket 36 are exposed to UV-C light. As the organic, or carbon based microorganisms on the surfaces of such items are exposed to the UV-C light, the molecular bonds in such microorganisms are broken, causing genetic damage and preventing such organisms from reproducing, rendering them harmless. The ability of UV-C light to disable such microorganisms in this manner is directly related to intensity of UV-C light and exposure time. In one (not shown) embodiment, power button 22 is attached to a timer which enables the cleaning cycle to proceed for a pre-set period of time. After the time period ends, UV-C light source 50 ceases emitting light and motor 48 turns off. A cleaning cycle of fifteen minutes has been found to be sufficient to insure sterilization of six small items. There could also be a relay circuit (not shown) which is preset for the appropriate sterilization cycle.

What has been illustrated and described herein is a sterilizer for small items. While the invention has been illustrated and described with reference to certain preferred embodiments, the present invention is not limited thereto. In particular, the foregoing specification and embodiments are intended to be illustrative and are not to be taken as limiting. Thus, alternatives such as structural or mechanical equivalents and modifications will become apparent to those skilled in the art upon reading the foregoing description. Accordingly, such alternatives, changes, and modifications are to be considered as forming a part of the present invention insofar as they fall within the spirit and scope of the appended claims.

1. A sterilizer comprising:
a housing forming an enclosure:
a receptacle movably arranged in said housing, said receptacle permitting the passage of UV-C radiation therethrough;
a source of UV-C radiation located within said enclosure;
and means for rotating said receptacle inside said enclosure;
wherein UV-C radiation from said source of UV-C radiation is directed through said receptacle as said receptacle is rotated.

2. The sterilizer of claim 1 wherein said means for rotating said receptacle is an electric motor.

3. The sterilizer of claim 1 wherein said receptacle is a basket which has openings sufficient to permit UV-C radiation to pass through to achieve sterilization in an optimum amount of time.

4. The sterilizer of claim 3 wherein the optimum amount of time is about fifteen minutes.

5. The sterilizer of claim 1 further comprising a door sealing said enclosure.

6. The sterilizer of claim 5 where said door has a window therein.

7. The sterilizer of claim 1 wherein said receptacle has means therein for agitating items placed in said receptacle.

8. The sterilizer of claim 1 wherein said UV-C radiation source emits radiation having a bandwidth between 200 nm and 290 nm.

9. The sterilizer of claim 1 wherein said UV-C radiation source emits radiation having a bandwidth between 250 nm and 260 nm.

10. The sterilizer of claim 1 further comprising a power switch operated by a timer.

11. The sterilizer of claim 1 further comprising a base mounting said housing.

12. The sterilizer of claim 1 wherein items placed in said receptacle are exposed to UV-C radiation and thereby sterilized.

13. A sterilizer for disinfecting items comprising:
an enclosure which can be opened and closed on at least one side;
a receptacle for receiving said items and movably arranged inside said enclosure wherein said receptacle does not interfere with the transmission of UV-C radiation;
a source of UV-C radiation located within said enclosure;
means for rotating said receptacle inside said enclosure;
a power source; and
a power switch electrically connected to said power source, said source of UV-C radiation and said means for rotating;
whereby when said sterilizer is connected to said power source and said power switch is activated, UV-C radiation from said source of UV-C radiation is uniformly directed onto said items located in said receptacle and said means for rotating causes said receptacle to move.

14. The sterilizer of claim 13 wherein exposure time of said items to said UV-C radiation is about fifteen minutes.
